(19) United States
(12) Patent Application Publication
(10) Pub. No.: US 2024/0369528 A1
(43) Pub. Date: Nov. 7, 2024

(54) PERFORMANCE TESTING METHOD AND SYSTEM FOR POLYCARBOXYLATE SUPERPLASTICIZER IN CONCRETE SYSTEM

(71) Applicant: QINGDAO UNIVERSITY OF TECHNOLOGY, Shandong (CN)

(72) Inventors: Dongshuai HOU, Shandong (CN); Muhan WANG, Shandong (CN); Xiang JI, Shandong (CN); Pan WANG, Shandong (CN); Xinpeng WANG, Shandong (CN); Yue ZHANG, Shandong (CN)

(21) Appl. No.: 18/030,180

(22) PCT Filed: Feb. 22, 2022
(86) PCT No.: PCT/CN2022/077162

§ 371 Date: Apr. 4, 2023

(30) Foreign Application Priority Data
Jan. 24, 2022 (CN) 202210078952.X

(51) Int. Cl. G01N 33/38; C04B 24/32; C04B 111/00

(52) U.S. Cl. CPC G01N 33/383; C04B 24/32; C04B 2111/00068

(57) ABSTRACT

The present disclosure provides a performance testing method and system for a polycarboxylate superplasticizer in a concrete system. An interface model is constructed based on a calcium silicate hydrate (C—S—H) gel model and a molecular dynamics model of a polycarboxylate superplasticizer, which can cover complexity of a cement particle interface and variability of the polycarboxylate superplasticizer, and can also establish a link between a microstructure of the polycarboxylate superplasticizer and a macroscopic fluidity of a cement across multiple scales. Meanwhile, friction resistance is accurately calculated based on the constructed interface model to accurately test a performance of the polycarboxylate superplasticizer, thereby shortening a screening cycle of the polycarboxylate superplasticizer and improving a performance optimization efficiency.

### CROSS REFERENCE TO RELATED APPLICATION

This application claims priority to the Chinese Patent Application No. 202210078952.X, filed with the China National Intellectual Property Administration (CNIPA) on Jan. 24, 2022, and entitled “PERFORMANCE TESTING METHOD AND SYSTEM FOR POLYCARBOXYLATE SUPERPLASTICIZER IN CONCRETE SYSTEM”, which is incorporated herein by reference in its entirety.

### TECHNICAL FIELD

The present disclosure relates to the technical field of concrete performance evaluation and concrete admixture, in particular to a performance testing method and system for a polycarboxylate superplasticizer in a concrete system.

### BACKGROUND

In recent years, with the rapid development of concrete technology, water reducer products have also been continuously updated and upgraded. The third-generation high-efficiency superplasticizer represented by polycarboxylate superplasticizer has many excellent properties and comprehensive performances. The polycarboxylate superplasticizer can greatly improve the workability of fresh concrete, which is also widely used in preparing concrete with a high-strength performance. Polycarboxylate superplasticizer is an important admixture in modern concrete technology. As a high-efficiency water reducer in the cement industry, the polycarboxylate superplasticizer is mainly used to reduce a water-cement ratio and control a setting time without losing fluidity. At present, polycarboxylate superplasticizer is the only high-efficiency superplasticizer that can maintain desirable fluidity of concrete after reaching a water-binder ratio of 2.0:moreover, the polycarboxylate superplasticizer can maintain the fluidity of concrete for a longer time than the traditional naphthalene sulfonate water reducer. Accordingly, polycarboxylate superplasticizers are widely used in high-performance concrete materials such as self-compacting concrete and ultra-high performance concrete.

In the prior art, there are some experimental studies on the fluidity of concrete mixed with polycarboxylate superplasticizers. These traditional research methods include slump test, Vebe Consistometer test, jumping table test, remodeling test, and deformation test. However, the traditional experimental method must acquire polycarboxylate superplasticizer molecules before testing, and is impossible to pre-test the performance without acquiring the polycarboxylate superplasticizer molecules. Moreover, these methods have long test time and cumbersome test steps, making the performance evaluation cycle of polycarboxylate superplasticizers long, which is not conducive to efficient design.

### SUMMARY

In order to solve the above problems in the prior art, an objective of the present disclosure is to provide a performance testing method and system for a polycarboxylate superplasticizer in a concrete system.

To achieve the above objective, the present disclosure provides the following technical solutions:

The present disclosure provides a performance testing method for a polycarboxylate superplasticizer in a concrete system, including the following steps:

constructing an interface model of a cement paste based on a calcium silicate hydrate (C—S—H) gel model and a molecular dynamics model of a polycarboxylate superplasticizer: setting a first end of the C—S—H gel model in the interface model as a first rigid body, and setting a second end of the C—S—H gel model in the interface model as a second rigid body:

setting molecular dynamics simulation parameters including a temperature, a time step, and a rigid body thickness:

assigning the molecular dynamics simulation parameters to the interface model, and conducting simulation based on a first preset condition to obtain an interface model of the cement paste under a standard atmospheric pressure:

assigning the molecular dynamics simulation parameters to the interface model of the cement paste under the standard atmospheric pressure, and conducting simulation based on a second preset condition to obtain a coordinate of a first side atom in the second rigid body on an x-axis of a space coordinate system: where the space coordinate system uses a boundary point at one end of a bottom of the second rigid body as an origin:

determining an interface friction according to the coordinate; and
determining a performance of the polycarboxylate superplasticizer in concrete according to the interface friction.

Preferably, a process of constructing the interface model of the cement paste based on the C—S—H gel model and the molecular dynamics model of the polycarboxylate superplasticizer specifically includes:

conducting supercell construction on an analogue of a C—S—H gel to obtain the C—S—H gel model: removing an intermediate silicon chain layer of the C—S—H gel model to obtain a C—S—H gel model with an intermediate defect space; and

embedding the molecular dynamics model of the polycarboxylate superplasticizer and a water molecule model into the intermediate defect space of the C—S—H gel model.

Preferably, a process of assigning the molecular dynamics simulation parameters to the interface model, and conducting simulation based on the first preset condition to obtain the interface model of the cement paste under the standard atmospheric pressure specifically includes:

assigning the molecular dynamics simulation parameters to the interface model to obtain a first interface model; and

fixing the second rigid body in the first interface model along a z-axis of the space coordinate system, applying a constant normal load with a preset value on the first rigid body of the first interface model, and conducting simulation to obtain the interface model of the cement paste at the standard atmospheric pressure.

Preferably, a process of assigning the molecular dynamics simulation parameters to the interface model of the cement paste under the standard atmospheric pressure, and conducting simulation based on the second preset condition to obtain the coordinate of the first side atom in the second rigid body on the x-axis of the space coordinate system specifically includes:

assigning the molecular dynamics simulation parameters to the interface model of the cement paste under the standard atmospheric pressure to obtain a second interface model; and

moving the first rigid body in the second interface model at a preset speed along the x-axis of the space coordinate system, conducting simulation on a shearing motion of cement particles using the second rigid body in the second interface model and a spring, and recording the coordinate of the first side atom in the second rigid body on the x-axis of the space coordinate system during the shearing motion.

Preferably, the C—S—H gel model has a size of 33.48 Å×29.56 Å×91.08 Å.

Preferably, the polycarboxylate superplasticizer is one selected from the group consisting of a methoxypolyethylene glycol monomethyl ether (MPEG) polycarboxylate superplasticizer, a methylallyl alcohol polyoxyethylene ether (TPEG) polycarboxylate superplasticizer, an isobutenol polyoxyethylene ether (HPEG) polycarboxylate superplasticizer, a 4-hydroxybutylvinyl polyoxyethylene ether (VPEG) polycarboxylate superplasticizer, and an allyl polyoxyethylene ether (APEG) polycarboxylate superplasticizer.

Preferably, the intermediate defect space of the C—S—H gel model has a size of 30 Å to 50 Å.

According to the specific embodiments provided by the present disclosure, the present disclosure discloses the following technical effects:

The present disclosure provides a performance testing method for a polycarboxylate superplasticizer in a concrete system. An interface model is constructed based on a calcium silicate hydrate (C—S—H) gel model and a molecular dynamics model of a polycarboxylate superplasticizer, which can properly cover complexity of a cement particle interface and variability of the polycarboxylate superplasticizer, and can also establish a link between a microstructure of the polycarboxylate superplasticizer and a macroscopic fluidity of a cement across multiple scales. Meanwhile, friction resistance of the interface is accurately calculated based on the constructed interface model to accurately test a performance of the polycarboxylate superplasticizer, thereby shortening a screening cycle of the polycarboxylate superplasticizer and improving performance optimization efficiency. In addition, a specific action process of the polycarboxylate superplasticizer in the cement is revealed from a microscopic scale, which is helpful to understand the mechanism of an influence of the polycarboxylate superplasticizer on a working performance of the cement. Therefore, the present disclosure provides a theoretical support for design and optimization of a molecular structure of the polycarboxylate superplasticizer, and can further provide guidance for experimental development or production of the polycarboxylate superplasticizer.

Corresponding to the performance testing method for a polycarboxylate superplasticizer in a concrete system, the present disclosure further provides a performance testing system for a polycarboxylate superplasticizer in a concrete system, including the following modules:

an interface model construction module configured to construct an interface model of a cement paste based on a C—S—H gel model and a molecular dynamics model of a polycarboxylate superplasticizer: set a first end of the C—S—H gel model in the interface model as a first rigid body, and set a second end of the C—S—H gel model in the interface model as a second rigid body:

a simulation parameter setting module configured to set molecular dynamics simulation parameters including a temperature, a time step, and a rigid body thickness:

a simulation module configured to assign the molecular dynamics simulation parameters to the interface model, and conduct simulation based on a first preset condition to obtain an interface model of the cement paste under a standard atmospheric pressure:

a coordinate determination module configured to assign the molecular dynamics simulation parameters to the interface model of the cement paste under the standard atmospheric pressure, and conduct simulation based on a second preset condition to obtain a coordinate of a first side atom in the second rigid body on an x-axis of a space coordinate system: where the space coordinate system uses a boundary point at one end of a bottom of the second rigid body as an origin:

an interface friction determination module configured to determine an interface friction according to the coordinate; and

a performance determination module configured to determine a performance of the polycarboxylate superplasticizer in concrete according to the interface friction.

In the present disclosure, technical effects achieved by the performance testing system for a polycarboxylate superplasticizer in a concrete system is the same as those achieved by the performance testing method for a polycarboxylate superplasticizer in a concrete system, which are not repeated here.

### BRIEF DESCRIPTION OF THE DRAWINGS

To describe the technical solutions in the embodiments of the present disclosure or in the prior art more clearly, the accompanying drawings required for the embodiments are briefly described below. Apparently, the accompanying drawings in the following description show merely some embodiments of the present disclosure, and a person of ordinary skill in the art may still derive other accompanying drawings from these accompanying drawings without creative efforts.

FIG. 1 shows a flowchart of a performance testing method for a polycarboxylate superplasticizer in a concrete system provided by the present disclosure;

FIG. 2 shows a schematic diagram of an interface model provided by an example of the present disclosure:

FIG. 3 shows a schematic diagram of a first molecular simulation process provided by the example of the present disclosure:

FIG. 4 shows a schematic diagram of a second molecular simulation process provided by the example of the present disclosure:

FIG. 5 shows a monomer chemical structure diagram of a methoxypolyethylene glycol monomethyl ether (MPEG) polycarboxylate superplasticizer provided by the example of the present disclosure:

FIG. 6 shows a test diagram of an interface friction provided by the example of the present disclosure:

FIG. 7 shows a test diagram of an average interface friction provided by the example of the present disclosure; and

FIG. 8 shows a schematic structural diagram of a performance testing system for a polycarboxylate superplasticizer in a concrete system provided by the present disclosure.

### DETAILED DESCRIPTION OF THE EMBODIMENTS

The technical solutions of the embodiments of the present disclosure are clearly and completely described below with reference to the accompanying drawings. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

An objective of the present disclosure is to provide a performance testing method and system for a polycarboxylate superplasticizer in a concrete system. The present disclosure intends to accurately reflect an influence of the polycarboxylate superplasticizer on the fluidity of concrete, thereby accurately evaluating a performance of the polycarboxylate superplasticizer, shortening a screening cycle of the polycarboxylate superplasticizer, and improving an efficiency of performance optimization.

To make the above-mentioned objective, features, and advantages of the present disclosure clearer and more comprehensible, the present disclosure will be further described in detail below in conjunction with the accompanying drawings and specific embodiments.

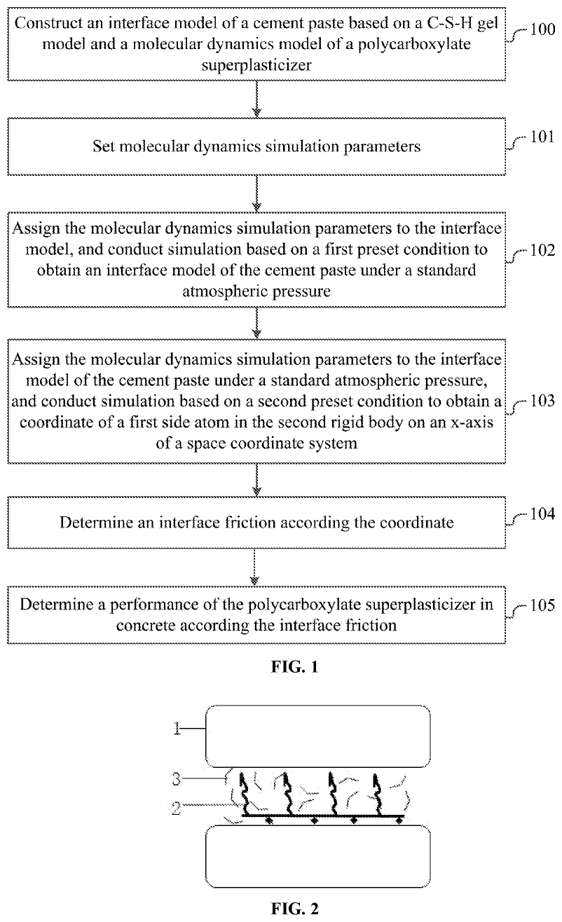

As shown in FIG. 1, a performance testing method for a polycarboxylate superplasticizer in a concrete system includes the following steps:

Step 100: an interface model of a cement paste is constructed based on a C—S—H gel model and a molecular dynamics model of a polycarboxylate superplasticizer. As shown in FIG. 2, the interface model includes a C—S—H gel model with an intermediate defect space, and the molecular dynamics model of the polycarboxylate superplasticizer and a water molecule model that are embedded in the intermediate defect space of the C—S—H gel model. For example, 11 Å tobermorite is selected as an analogue of C—S—H gels. The C—S—H gel model is obtained by conducting supercell construction of a unit cell of the 11 Å tobermorite along axes a, b, and c. As a specific example, the C—S—H gel model may specifically have a size of 33.48 Å×29.56 Å×91.08 Å. This C—S—H gel model has a suitable size, is easy to modify, and is convenient to fill with polycarboxylate superplasticizer and water molecules, such that it is conducive to better simulating the cement particle interface to form a more accurate interface model.

In an example of the present disclosure, in step 100, the C—S—H gel model with an intermediate defect space can be constructed according to the following method: conducting supercell construction on an analogue of a C—S—H gel to obtain the C—S—H gel model: removing an intermediate silicon chain layer of the C—S—H gel model to obtain a C—S—H gel model with an intermediate defect space. Removing the intermediate silicon chain layer preferably includes: removing four layers of silicon chains located in an intermediate space of the C—S—H gel model and Ca atoms and water molecules having a chemical coordination relationship with each silicon chain.

In a specific example of the present disclosure, the method further includes preferably: removing a bottom silicon chain layer and a top silicon chain layer of the C—S—H gel model with an intermediate defect space. Removing the bottom silicon chain layer preferably includes: removing one layer of a silicon chain located in a bottom space of the C—S—H gel model and Ca atoms and water molecules having a chemical coordination relationship with each silicon chain. Removing the top silicon chain layer preferably includes: removing one layer of a silicon chain located in a top space of the C—S—H gel model and Ca atoms and water molecules having a chemical coordination relationship with each silicon chain.

In step 100, the interface model can be obtained by implementing the following steps: embedding the molecular dynamics model of the polycarboxylate superplasticizer and a water molecule model into the intermediate defect space of the C—S—H gel model.

In an example of the present disclosure, the molecular dynamics model of the polycarboxylate superplasticizer is obtained by: a monomer molecular structure of the polycarboxylate superplasticizer is drawn according to a monomer chemical structure of the polycarboxylate superplasticizer, and monomer molecules of the polycarboxylate superplasticizer are polymerized to construct a molecular structure of the polycarboxylate superplasticizer: by a Forcite Tools module in Materials Studios software, molecular dynamics optimization is conducted on the molecular structure of the polycarboxylate superplasticizer to obtain the molecular dynamics model of the polycarboxylate superplasticizer.

In an example of the present disclosure, the molecular dynamics model of the polycarboxylate superplasticizer is one selected from the group consisting of a molecular dynamics model of a methoxypolyethylene glycol monomethyl ether (MPEG) polycarboxylate superplasticizer (PCE) (as shown in FIG. 5), a molecular dynamics model of a methylallyl alcohol polyoxyethylene ether (TPEG-PCE) polycarboxylate superplasticizer, a molecular dynamics model of an isobutenol polyoxyethylene ether (HPEG-PCE) polycarboxylate superplasticizer, a molecular dynamics model of a 4-hydroxybutylvinyl polyoxyethylene ether (VPEG-PCE) polycarboxylate superplasticizer, and a molecular dynamics model of an allyl polyoxyethylene ether (APEG-PCE) polycarboxylate superplasticizer.

In an example of the present disclosure, the number of polycarboxylate superplasticizer molecules in the molecular dynamics model of the polycarboxylate superplasticizer is adapted to the size of the intermediate defect space of the C—S—H gel model with an intermediate defect space.

In this example of the present disclosure, there is no special requirement on a method for constructing the water molecule model.

In an example of the present disclosure, an embedding quantity of the water molecule model is determined according to a water content of the formed interface model.

In an example of the present disclosure, the intermediate defect space of the C—S—H gel model with an intermediate defect space has a size of 30 Å to 50 Å. Controlling the defect size is beneficial to the filling of the molecular dynamics model of the polycarboxylate superplasticizer and the water molecule model, so as to accurately simulate the situation of the polycarboxylate superplasticizer at the interface between cement particles.

Step 101: molecular dynamics simulation parameters are set. The molecular dynamics simulation parameters include a temperature, a time step, and a rigid body thickness. For example, under a temperature of 298 K, by a Berendsen method as a temperature control method, with a time step set to 1 fs, a top of the C—S—H gel model in the interface model is set as a first rigid body, and a bottom of the C—S—H gel model in the interface model is set as a second rigid body: the first rigid body and the second rigid body each have a thickness set to 4.6 Å.

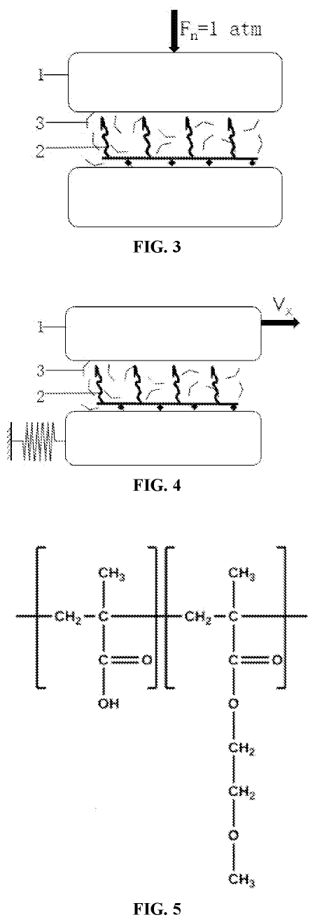

Step 102: the molecular dynamics simulation parameters are assigned to the interface model, and simulation based is conducted on a first preset condition to obtain an interface model of the cement paste under a standard atmospheric pressure. For example, after assigning the molecular dynamics simulation parameters to the interface model, the second rigid body is fixed along a z-axis direction, and a constant normal load of 1 atm is applied to the first rigid body along a negative direction of the z-axis (as shown in FIG. 3), where the normal load acts uniformly on a surface of the first rigid body, simulating a state of the cement paste under a standard atmospheric pressure. This process is mainly based on the molecular dynamics simulation of the interface model based on lammps software. In the present disclosure, various methods can be used to conduct molecular dynamics simulation on the interface model, preferably using the lammps software to conduct the molecular dynamics simulation on the interface model, showing a high simulation degree, excellent calculation efficiency, and accurate and reliable results.

Step 103: molecular dynamics simulation parameters are assigned to the interface model of the cement paste under the standard atmospheric pressure, and simulation is conducted based on a second preset condition to obtain a coordinate of a first side atom in a second rigid body on an x-axis of a space coordinate system. The space coordinate system uses a boundary point at a front end of a bottom of the second rigid body as an origin. For example, after assigning molecular dynamics simulation parameters to the interface model of cement paste under the standard atmospheric pressure, the first rigid body is set to a constant velocity along a positive direction of the x-axis, and the other parts of the C—S—H gel model are free to move: the second rigid body is connected to a spring with a fixed stiffness coefficient of 0.001 N/m (as shown in FIG. 4) to simulate a shearing motion of cement particles: a coordinate of the leftmost atom in the lower rigid body in the x-axis direction is recorded during the shearing motion. For molecular simulations, the constant velocity is set to 1 m/s. When conducting the molecular simulation, the constant velocity is set to 1 m/s, and the movement of the upper rigid body drives the movement of the lower parts to more realistically simulate the relative movement of cement particles in the cement paste, thereby improving the accuracy and precision of simulation test. In addition, a spring force to characterize the interface friction can well calculate the spring force during the shearing, that is, the interface friction during the shearing can be obtained, showing convenient calculation, desirable accuracy, and high efficiency. In FIG. 2 to FIG. 4, 1 is the C—S—H gel model, 2 is the polycarboxylate superplasticizer model, and 3 is the water molecule model.

Step 104: an interface friction is determined according to the coordinate. Specifically, the spring force F during the shearing can be calculated through the coordinate of the leftmost atom in the x-axis direction, that is, the interface friction, where the calculation formula of F is as follows:

$$F = - kx ; and \qquad ( 1 )$$

$$x = {x}_{n} - {x}_{o ;} \qquad ( 2 )$$

where

k is a stiffness coefficient of the spring, xₙ is the coordinate of the leftmost atom in the x-axis direction, and xₒ is an original coordinate of the leftmost atom in the x-axis direction.

Step 105: a performance of the polycarboxylate superplasticizer in concrete is determined according to the interface friction. Specifically, an interface friction curve during the shearing of the interface model is obtained based on the interface friction: by comparing the magnitude of the interface friction, the performance is evaluated for the polycarboxylate superplasticizer in concrete. Before obtaining the interface friction curve, Origin software is optionally selected to process the obtained interface friction data: this data processing method has simple operations, a high efficiency, accurate processing results, and convenient use.

An example is provided below to illustrate a specific implementation process of the performance testing method for a polycarboxylate superplasticizer in a concrete system provided above. In the actual application, parameters in the following examples are not specifically limited to the technical solutions provided above in the present disclosure.

1) A 11 Å tobermorite unit cell was derived from a structure database of Materials Studios simulation software, and the tobermorite unit cell was subjected to supercell construction along axes a, b, and c by 4 times, 3 times, and 2 times, respectively, to obtain a=33.48 Å, b=29.56 Å, and c=91.08 Å, namely the C—S—H gel model. According to a monomer chemical structure of MPEG-PCE, a monomer molecular structure of the MPEG-PCE was drawn: polymerization and dynamic optimization were conducted on 4 MPEG-PCE monomer molecular structures, to obtain a molecular dynamics model of the MPEG-PCE. A water molecule model was constructed based on a water molecular formula.

2) The top and bottom layers of silicon chains and the nearby Ca atoms and water molecules in the C—S—H gel model were removed, and then the intermediate four layers of silicon chains and the nearby Ca atoms and water molecules in the C—S—H gel model were removed, such that a defect was formed in the middle of the C—S—H gel model. By modifying Ca on the surface of the model, two MPEG-PCE models and 100, 200, and 300 water molecule models were filled in the defect to construct an interface model.

3) The molecular dynamics simulation parameters were set: a canonical ensemble was selected as a simulation ensemble, a temperature was 298 K, a Berendsen method was selected as a temperature control method, a time step was 1 fs: in the interface model, an upper part of a top C—S—H gel model and a lower part of a bottom C—S—H gel model were separately set as a rigid body with a thickness of 4.6 Å.

4) The simulation parameters set in step 3) were assigned to each component of the interface model in step 2), where the lower rigid body was fixed along a z-axis direction, the upper rigid body acted on a constant normal load of 1 atm along a negative direction of the z-axis, and the normal load acted uniformly on the surface of the upper rigid body; and a state of a cement paste was simulated under a standard atmospheric pressure, with a simulation time set to Ins.

5) The simulation parameters set in step 3) were assigned to each component of the interface model obtained by the final simulation in step 4), where the upper rigid body was set at a constant speed along a positive direction of the x-axis, the other parts of the C—S—H gel model were free to move, and the lower rigid body was connected to a spring with a fixed stiffness coefficient of 0.001 N/m: a shearing motion of cement particles was simulated, and a coordinate of the leftmost atom in the lower rigid body in the x-axis direction was recorded during the shearing motion, with a simulation time set to 2 ns.

6) A spring force F during the shearing could be calculated through the coordinate of the leftmost atom in the x-axis direction, that is, the interface friction, where the calculation formula of F is as shown in equations (1) and (2).

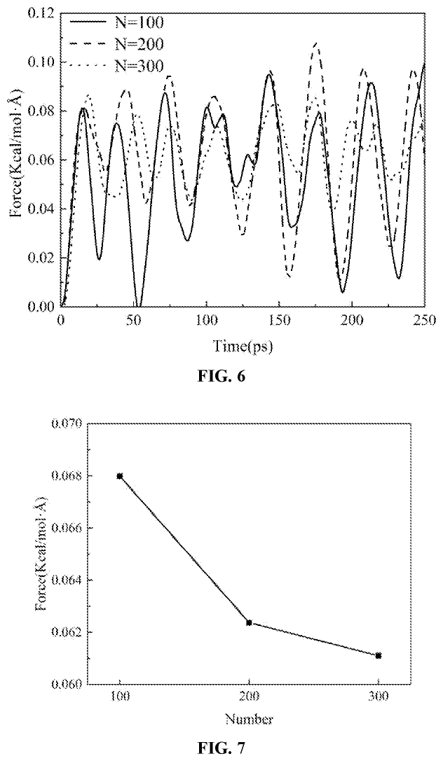

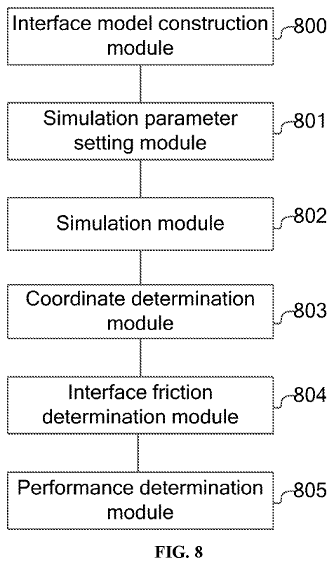

7) An interface friction curve (FIG. 6) during the shearing of the interface model was obtained based on processing the data obtained in step 6): by comparing the magnitude of the interface friction, the performance was evaluated for the polycarboxylate superplasticizer in concrete (FIG. 7).

Based on the above description, the performance testing method for a polycarboxylate superplasticizer in a concrete system is a simulation test and evaluation method for performances of the polycarboxylate superplasticizer in the concrete system by a computer simulation technology, as a design method based on molecular dynamics simulation. The interface model can properly cover complexity of a cement particle interface and variability of the polycarboxylate superplasticizer, and can also establish a link between a microstructure of the polycarboxylate superplasticizer and a macroscopic fluidity of a cement across multiple scales. By accurately calculating the frictional resistance at the interface, the method can be further applied to evaluate the performance of polycarboxylate superplasticizer. In the present disclosure, an action process of the polycarboxylate superplasticizer in the cement is revealed from a microscopic scale, which is helpful to understand the mechanism of an influence of the polycarboxylate superplasticizer on a working performance of the cement. Therefore, the present disclosure provides a theoretical support for design and optimization of a molecular structure of the polycarboxylate superplasticizer, and can further provide a guidance for experimental development or production of the polycarboxylate superplasticizer.

In addition, corresponding to the performance testing method for a polycarboxylate superplasticizer in a concrete system, the present disclosure further provides a performance testing system for a polycarboxylate superplasticizer in a concrete system. As shown in FIG. 8, the system includes: an interface model construction module 800, a simulation parameter setting module 801, a simulation module 802, a coordinate determination module 803, an interface friction determination module 804, and a performance determination module 805.

The interface model construction module 800 is configured to construct an interface model of a cement paste based on a C—S—H gel model and a molecular dynamics model of a polycarboxylate superplasticizer.

The simulation parameter setting module 801 is configured to set molecular dynamics simulation parameters. The molecular dynamics simulation parameters include a temperature, a time step, and a rigid body thickness.

The simulation module 802 is configured to assign the molecular dynamics simulation parameters to the interface model, and conduct simulation based on a first preset condition to obtain an interface model of the cement paste under a standard atmospheric pressure.

The coordinate determination module 803 is configured to assign the molecular dynamics simulation parameters to the interface model of the cement paste under the standard atmospheric pressure, and conduct simulation based on a second preset condition to obtain a coordinate of a first side atom in the second rigid body on an x-axis of a space coordinate system. The space coordinate system uses a boundary point at one end of a bottom of the second rigid body as an origin.

The interface friction determination module 804 is configured to determine an interface friction according the coordinate.

The performance determination module 805 is configured to determine a performance of the polycarboxylate superplasticizer in concrete according the interface friction.

Each embodiment of the present specification is described in a progressive manner, each example focuses on the difference from other examples, and the same and similar parts between the examples may refer to each other. Since the system disclosed in an embodiment corresponds to the method disclosed in another embodiment, the description is relatively simple, and reference can be made to the method description.

Specific examples are used herein to explain the principles and embodiments of the present disclosure. The foregoing description of the embodiments is merely intended to help understand the method of the present disclosure and its core ideas; besides, various modifications may be made by a person of ordinary skill in the art to specific embodiments and the scope of application in accordance with the ideas of the present disclosure. In conclusion, the content of the present specification shall not be construed as limitations to the present disclosure.

1. A performance testing method for a polycarboxylate superplasticizer in a concrete system, the performance testing method comprising:
constructing an interface model of a cement paste based on a calcium silicate hydrate (C—S—H) gel model and a molecular dynamics model of a polycarboxylate superplasticizer;
setting a first end of the C—S—H gel model in the interface model as a first rigid body, and setting a second end of the C—S—H gel model in the interface model as a second rigid body;
setting molecular dynamics simulation parameters comprising a temperature, a time step, and a rigid body thickness;
assigning the molecular dynamics simulation parameters to the interface model, and conducting simulation based on a first preset condition to obtain an interface model of the cement paste under a standard atmospheric pressure;
assigning the molecular dynamics simulation parameters to the interface model of the cement paste under the standard atmospheric pressure, and conducting simulation based on a second preset condition to obtain a coordinate of a first side atom in the second rigid body on an x-axis of a space coordinate system; wherein the space coordinate system uses a boundary point at one end of a bottom of the second rigid body as an origin;
determining an interface friction according to the coordinate; and
determining a performance of the polycarboxylate superplasticizer in concrete according to the interface friction.

2. The performance testing method according to claim 1, wherein constructing the interface model of the cement paste based on the C—S—H gel model and the molecular dynamics model of the polycarboxylate superplasticizer specifically comprises:
conducting supercell construction on an analogue of a C—S—H gel to obtain the C—S—H gel model; removing an intermediate silicon chain layer of the C—S—H gel model to obtain a C—S—H gel model with an intermediate defect space; and
embedding the molecular dynamics model of the polycarboxylate superplasticizer and a water molecule model into the intermediate defect space of the C—S—H gel model.

3. The performance testing method according to claim 1, wherein assigning the molecular dynamics simulation parameters to the interface model, and conducting simulation based on the first preset condition to obtain the interface model of the cement paste under the standard atmospheric pressure specifically comprises:
assigning the molecular dynamics simulation parameters to the interface model to obtain a first interface model; and
fixing the second rigid body in the first interface model along a z-axis of the space coordinate system, applying a constant normal load with a preset value on the first rigid body of the first interface model, and conducting simulation to obtain the interface model of the cement paste at the standard atmospheric pressure.

4. The performance testing method according to claim 1, wherein assigning the molecular dynamics simulation parameters to the interface model of the cement paste under the standard atmospheric pressure, and conducting simulation based on the second preset condition to obtain the coordinate of the first side atom in the second rigid body on the x-axis of the space coordinate system specifically comprises:
assigning the molecular dynamics simulation parameters to the interface model of the cement paste under the standard atmospheric pressure to obtain a second interface model; and
moving the first rigid body in the second interface model at a preset speed along the x-axis of the space coordinate system, conducting simulation on a shearing motion of cement particles using the second rigid body in the second interface model and a spring, and recording the coordinate of the first side atom in the second rigid body on the x-axis of the space coordinate system during the shearing motion.

5. The performance testing method according to claim 1, wherein the C—S—H gel model has a size of 33.48 Å×29.56 Å×91.08 Å.

6. The performance testing method according to claim 2, wherein the polycarboxylate superplasticizer is one-selected from the group consisting of a methoxypolyethylene glycol monomethyl ether (MPEG) polycarboxylate superplasticizer, a methylallyl alcohol polyoxyethylene ether (TPEG) polycarboxylate superplasticizer, an isobutenol polyoxyethylene ether (HPEG) polycarboxylate superplasticizer, a 4-hydroxybutylvinyl polyoxyethylene ether (VPEG) polycarboxylate superplasticizer, and an allyl polyoxyethylene ether (APEG) polycarboxylate superplasticizer.

7. The performance testing method according to claim 2, wherein the intermediate defect space has a size of 30 Å to 50 Å.

8. A performance testing system for a polycarboxylate superplasticizer in a concrete system, the performance testing system comprising:
an interface model construction module configured to construct an interface model of a cement paste based on a C—S—H gel model and a molecular dynamics model of a polycarboxylate superplasticizer; set a first end of the C—S—H gel model in the interface model as a first rigid body, and set a second end of the C—S—H gel model in the interface model as a second rigid body;
a simulation parameter setting module configured to set molecular dynamics simulation parameters comprising a temperature, a time step, and a rigid body thickness;
a simulation module configured to assign the molecular dynamics simulation parameters to the interface model, and conduct simulation based on a first preset condition to obtain an interface model of the cement paste under a standard atmospheric pressure;
a coordinate determination module configured to assign the molecular dynamics simulation parameters to the interface model of the cement paste under the standard atmospheric pressure, and conduct simulation based on a second preset condition to obtain a coordinate of a first side atom in the second rigid body on an x-axis of a space coordinate system; wherein the space coordinate system uses a boundary point at one end of a bottom of the second rigid body as an origin;
an interface friction determination module configured to determine an interface friction according to the coordinate; and
a performance determination module configured to determine a performance of the polycarboxylate superplasticizer in concrete according to the interface friction.
